(19) United States
(12) Patent Application Publication
(10) Pub. No.: US 2009/0247826 A1
(43) Pub. Date: Oct. 1, 2009

(54) TUBE FOR ENDOSCOPE

(75) Inventor: Takeshi KIDA, Kunitachi-shi (JP)

Correspondence Address: Frishauf, Holtz Goodman & Chick, P.C., 16th Floor, 220 Fifth Avenue, New York, NY, 10001-7708, US

(73) Assignee: Olympus Corporation, Tokyo (JP)

(21) Appl. No.: 12/364,641

(22) Filed: Feb. 3, 2009

Related U.S. Application Data
(60) Provisional application No. 61/044,163, filed on Apr. 11, 2008.

(30) Foreign Application Priority Data
Mar. 28, 2008 (JP) 2008-087908
Jan. 30, 2009 (JP) 2009-020083

(51) Int. Cl. A61B 1/00

(52) U.S. Cl. 600/121

(57) ABSTRACT

There is provided a tube for an endoscope, including a tubular member forming a conduit line, a net layer which covers an outside of the tubular member and is formed of a hard linear member, and a covering layer which covers an outside of the net layer, wherein the covering layer is formed of a material includes thermoplastic resin, and has a thickness ranging from 0.02 to 0.5 mm.

### CROSS-REFERENCE TO RELATED APPLICATIONS

This application claims the benefit of U.S. Provisional Application No. 61/044,163, filed Apr. 11, 2008.

This application is based upon and claims the benefit of priority from prior Japanese Patent Applications No. 2008-087908, filed Mar. 28, 2008; and No. 2009-020083, filed Jan. 30, 2009, the entire contents of both of which are incorporated herein by reference.

### BACKGROUND OF THE INVENTION

1. Field of the Invention

The present invention relates to a tube used in an endoscope.

2. Description of the Related Art

In an endoscope, tubes such as a surgical instrument insertion channel, an air feed tube, and a water feed tube are used. These tubes are built in a main body of the endoscope from operation part to an end of an insertion part. Since the endoscope is repeatedly bent in use, these tubes are subjected to external pressure. Therefore, these tubes are required to have excellent durability. In the surgical instrument insertion channel disclosed in Jpn. Pat. Appln. KOKAI Publication No. 3-205022, a tube is covered by a net formed of a hard wire rod, and a covering layer is further formed on the outside of the net.

### BRIEF SUMMARY OF THE INVENTION

A tube built in an endoscope is not fixed in a flexible tube part of an insertion part. Therefore, the tube is in contact with other tubes and other components such as a wire coil, and thus they rub against each other. A covering layer of the tube is worn away due to the contact with other components and the rubbing. A net layer under the covering layer may eventually be exposed. In particular, when the tube rubs against other components in such a state that the net layer is exposed, the other components may be damaged.

An object of the invention is to provide a tube for an endoscope which realizes enhancement of wear resistance of a covering layer and has excellent durabilty.

In order to achieve the above object, the present invention provides a tube for an endoscope, comprising: a tubular member forming a conduit line; a net layer which covers an outside of the tubular member and is formed of a hard linear member; and a covering layer which covers an outside of the net layer, wherein the covering layer is formed of a material comprising thermoplastic resin, and has a thickness ranging from 0.02 to 0.5 mm.

The invention further allows the covering layer to be formed of a material comprising thermoplastic resin and inorganic powder and/or silicone oil. In addition, an adhesive layer and/or a surface-treated layer can be further provided between the net layer and the covering layer.

In another aspect of the present invention, there is provided a tube for an endoscope in which the net layer is formed of a hard linear member having an elliptical cross section, in which a diameter direction of a tubular member is the minor axis.

In addition, an endoscope having the tube for an endoscope of the invention is provided.

According to the invention the wear resistance of the covering layer is enhanced, whereby the tube for an endoscope having excellent durability can be provided.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic view showing a partial outline of an endoscope body;

FIG. 2 is a cutaway view illustrating the tube for an endoscope according to one embodiment of the present invention;

FIG. 3 is a partial cross section view illustrating the tube for an endoscope according to one embodiment;

FIG. 4 is a partial cross section view illustrating the tube for an endoscope according to another embodiment;

FIG. 5 is a partial cross section view illustrating the tube for an endoscope according to still another embodiment;

FIG. 6 is a partial cross section view illustrating the tube for an endoscope according to yet another embodiment;

FIG. 7 is a partial cross section view illustrating the tube for an endoscope according to still yet another embodiment;

FIG. 8A is a front view of a tip part of the endoscope;

FIG. 8B is a cross-sectional view of the tip part along arrow A-A in FIG. 8A;

FIG. 8C is a cross-sectional view for illustrating connection parts between the tip part shown in FIG. 8B, a connector, and the tube for an endoscope;

FIG. 9 is a graph showing the evaluation result of thickness and durability of a covering layer;

FIG. 10A is a schematic view showing an evaluation device for measuring a bending force;

FIG. 10B is a schematic view showing the evaluation device at the time when the bending force is measured by the evaluation device of FIG. 10A; and

FIG. 11 is a graph showing the evaluation result of thickness of a covering layer and the bending force.

### DETAILED DESCRIPTION OF THE INVENTION

FIG. 1 is a partial schematic view of an endoscope body. An endoscope body 1 is composed mostly of an insertion part 2 inserted into a body cavity, an operation part 3, and a flexible cord 4. The insertion part 2 includes a flexible part, and the proximal end thereof is connected to the end of the operation part 3. The flexible cord 4 is connected to the proximal side surface of the operation part 3.

The Insertion part 2 is constituted of a corrugated tube part (flexible tube part) 6, a curved part 7 connected to the tip of the corrugated tube part 6, and a tip part 8 connected to the tip of the curved part 7. A curving operation wire is inserted into the interior from the insertion part 2 to the operation part 3. Tubes for an endoscope such as a surgical instrument insertion channel, an air feed tube, and a water feed tube are built in that interior space of the operation part 3 and the insertion part 2. The operation part 3 is provided with a surgical instrument insertion opening 5. A rear end side connector 9 to which the rear end of the tube for an endoscope is connected is provided in the interior adjacent to the surgical instrument insertion opening 5.

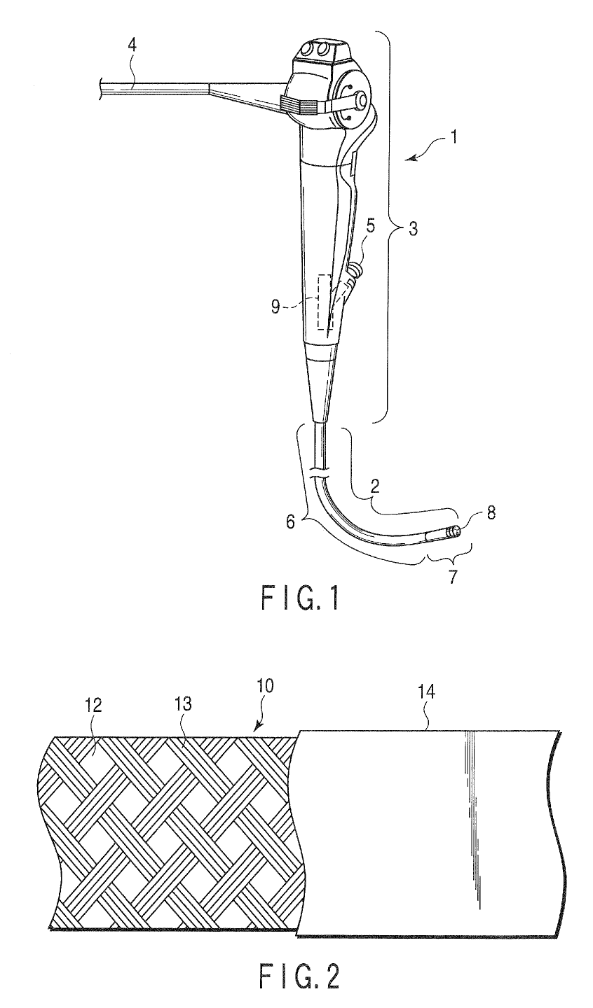

FIG. 2 is an external view of a partially fractured tube for an endoscope. A tube 10 for an endoscope has a tubular member 12 forming a conduit line, a net layer 13 for covering the outside of the tubular member 12, and a covering layer 14 for covering the outside of the net layer 13. The tubular member 12 is a conduit line formed of a material comprising thermoplastic resin, and may he a tube, for example. The net layer 13 is formed of a hard linear member formed into a net. Although the hard linear member is not limited thereto, a metal wire is suitably used.

The covering layer 14 is formed of a material comprising thermoplastic resin. As thermoplastic resin, resin well known in the art can be arbitrarily used, such as olefinic resin, nylon elastomer, styrene elastomer, polyester elastomer, acrylic elastomer, and polyurethane fluororesin elastomer. The covering layer 14 and the tubular member 12 may be formed of the same or different thermoplastic resin. The covering layer 14 and the tubular member 12 may be formed of only thermoplastic resin, or may be formed of a mixture composed of thermoplastic resin and other material.

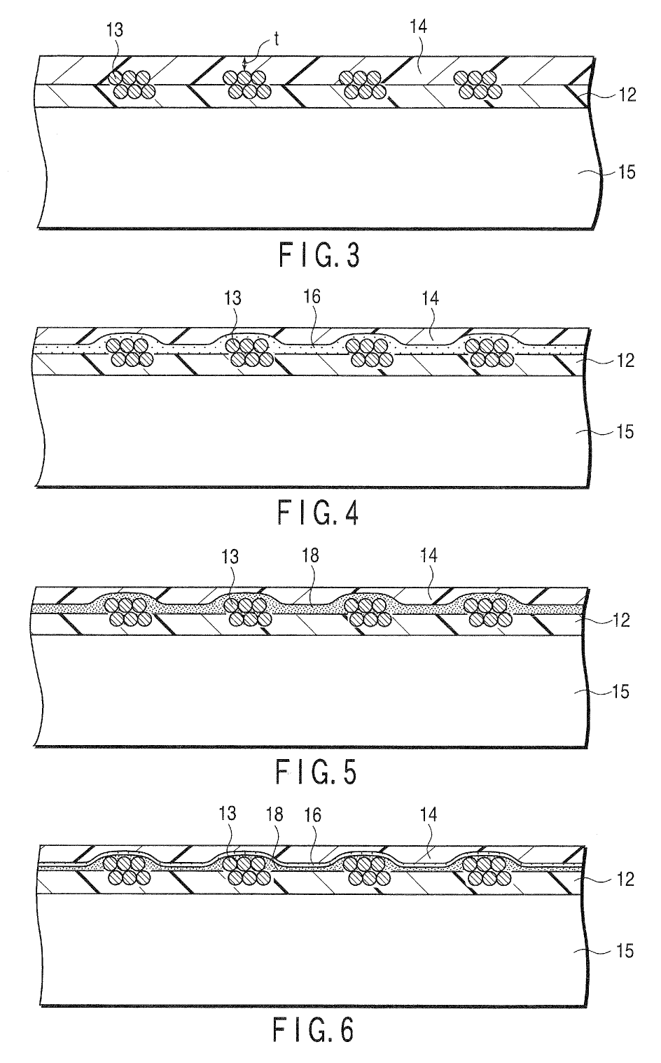

In a first embodiment of the invention, the covering layer 14 in the tube 10 for an endoscope has a thickness t ranging from 0.02 to 0.5 mm. FIG. 3 is a partially engreated sectional side view of the tube 10. In FIG. 3, a conduit line 15 is formed by the tubular member 12. The net layer 13 is formed outside the tubular member 12, and the covering layer 14 is formed outside the net layer 13. The thickness of the covering layer 14 is the thickness t from the top end of the net layer 13, as shown in FIG. 3, that is, the thickness of the portion of the covering layer 14 having the smallest thickness.

The covering layer 14 has the thickness t of 0.02 to 0.5 mm, whereby the surface of the covering layer 14 is planarized and thus to eliminate irregularities of the surface. As a result, the contact at a point is changed into a contact on a surface, whereby the wear resistance of the covering layer can be enhanced. If the thickness t is less than 0.02 mm, the surface of the covering layer becomes irregular, and a stress concentrates at the protrusions; therefore, the wear resistance of the covering layer is reduced, and the net layer is easily exposed due to the rubbing against other components. If the thickness t is not less than 0.5 mm, the outer diameter of the tube becomes greater. Further, if the thickness t is not less than 0.5 mm, the tube is hardened, reducing the curving performance. Therefore, while the operating force for bending a curved tube becomes greater, the operability of the endoscope is reduced. Thus, the thickness t of the covering layer is preferably 0.02 to 0.5 mm.

In a second embodiment of the invention, the covering layer 14 in the tube 10 for an endoscope is formed of a material comprising thermoplastic resin and inorganic powder. Since the covering layer formed of only thermoplastic resin is soft, the surface may exhibit undulation when being bent. If the surface is undulated, the frictional force in the rubbing against other tubes is increased, accelerating the wear of the covering layer. However, inorganic powder is mixed with thermoplastic resin in the formation of the covering layer to thereby smooth the surface of the covering layer, whereby the frictional force in the rubbing against other tubes is reduced. Thus, the wear resistance of the covering layer can be enhanced.

Inorganic powder used in the invention is not limited to, but it can be selected from barium sulfate, carbon-based powder, boron nitride (BN), molybdenum disulfide, silicon nitride, alumina, mica, and the mixture of them. Barium sulfate is especially preferably used. Powder having a particle diameter of not more than 5 μm is preferably used.

As the mixing ratio of thermoplastic resin and inorganic powder, 5 to 70 parts by weight of inorganic powder is mixed with 100 parts by weight of thermoplastic resin, and preferably 15 to 50 parts by weight of inorganic powder.

The tubular member 12 may be formed of the thermoplastic resin mixed with the inorganic powder.

In a third embodiment of the invention, the covering layer 14 in the tube 10 for an endoscope is formed of a material comprising thermoplastic resin and silicone oil. When silicone oil is mixed with thermoplastic resin in the formation of the covering layer, the silicone oil gradually oozes out of the covering layer, and an ultra-thin lubricating layer formed of silicone oil is formed on the surface of the covering layer. The effect of smoothing the surface of the covering layer can be obtained by virtue of the lubricating layer, whereby the slip properties of the surface of the covering layer are enhanced. According to this constitution, the frictional force in the rubbing against other tubes is reduced. Thus, the wear resistance of the covering layer can be enhanced.

Silicone oil with a viscosity ranging from 8500 to 1500000 CS is preferably used. If silicone oil with too low viscosity is used, the silicone oil easily leaches out, resulting in generation of stickiness of the covering layer. Further, silicone oil with too high viscosity is hard and fragile, and is difficult to mix with thermoplastic resin. Thus, the silicone oil with the viscosity ranging from 8500 to 1500000 CS is preferably used.

As the mixing ratio of thermoplastic resin and silicone oil, preferably 10 to 50 parts by weight of silicone oil is mixed with 100 parts by weight of thermoplastic resin, and more preferably 15 to 35 parts by weight of silicone oil.

The tubular member 12 may be formed of the thermoplastic resin mixed with the silicone oil.

Further, in a fourth embodiment of the invention, the covering layer 14 in the tube 10 for an endoscope is formed of a material comprising thermoplastic resin, inorganic powder, and silicone oil. The inorganic powder and the silicone oil are mixed with the thermoplastic resin in the formation of the covering layer, whereby the surface of the covering layer is smoothed and, at the same time, the slip properties thereof can be enhanced. According to this constitution, the wear resistance of the covering layer can be further enhanced.

A fifth embodiment of the invention is shown in FIG. 4. In this embodiment, the tube 10 for an endoscope is further provided with an adhesive layer 16 in the position between the net layer 13 and the covering layer 14. Thermoplastic resin forming the covering layer 14 may be peeled off from the net layer 13 when being subjected to external pressure. When the covering layer 14 is peeled off from the net layer 13, the outside of the covering layer 14 rubs against other tubes on its outer side and, at the same time, rubs against the net layer 13 on its inner side. In this case, the wear of the covering layer 14 is further accelerated. The peel-off of the covering layer 14 easily occurs especially when the tubular member 12 is formed of fluororesin or olefinic resin.

Thus, in this embodiment, the adhesive layer 16 is formed outside the net layer 13. The covering layer 14 is formed outside the adhesive layer 16. The adhesive strength between the net layer 13 and the covering layer 14 is increased by virtue of the adhesive layer 16, whereby the covering layer 14 becomes difficult to be peeled off from the net layer 13. Thus, the formation of the adhesive layer 16 realizes prevention of the peel-off of the covering layer 14, resulting in the enhancement of the wear resistance of the covering layer 14.

Although the adhesive agent forming the adhesive layer 16 is not limited to, an appropriate adhesive agent can be arbitrarily used, such as epoxy-based adhesive, polyimide-based adhesive, phenol-based adhesive, silicone-based adhesive, and acrylic-based adhesive.

As described above, when the tubular member 12 is formed of fluororesin or olefinic resin, the covering layer is easily peeled off; therefore, it is particularly effective that the adhesive layer is used in this case.

A sixth embodiment of the invention is shown in FIG. 5. In this embodiment, the tube 10 for an endoscope is further provided with a surface-treated layer 18 in the position between the net layer 13 and the covering layer 14. The surface-treated layer 18 is formed by applying the surface treatment on the surface of the net layer 13. Examples of the surface treatment include, but are not limited to, a treatment using a chemical liquid comprising aromatic alkali metal compound (for example, sodium-naphthalene), a so-called Tetra-Etch, plasma treatment, corona discharge, laser treatment, UV treatment, and itro treatment. As in the case of the adhesive layer, it is possible to prevent the covering layer 14 from being peeled off from the net layer 13 by virtue of the surface-treated layer 18, whereby the wear resistance of the covering layer 14 can be enhanced.

A seventh embodiment of the invention is shown in FIG. 6. The tube 10 for an endoscope is provided with the adhesive layer 16 and the surface-treated layer 18. The surface-treated layer 18 is formed on the net layer 13, and the adhesive layer 16 is formed on the surface-treated layer 18. According to this constitution, the peel-off of the net layer 13 and the covering layer 14 can be further prevented, whereby the wear resistance of the covering layer 14 can be enhanced.

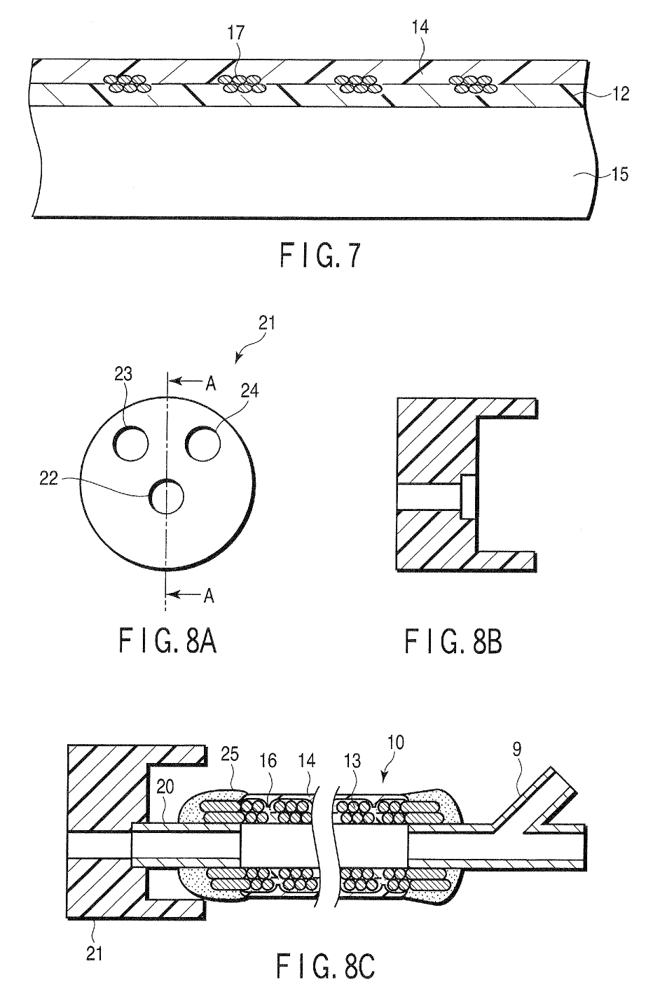

An eighth embodiment of the invention is shown in FIG. 7. In this embodiment, the tube 10 for an endoscope is provided with a net layer 17. The net layer 17 is formed of a hard linear member having an elliptical cross section, in which the diameter direction of the tubular member 12 is the minor axis. The thickness of the net layer 17 can be reduced by using a flattened hard linear member. The thickness of the covering layer 14 can be increased with decrease in thickness of the net layer 17. Namely, the thickness of the covering layer 14 can be increased while maintaining the outer diameter of the tube. Therefore, the wear resistance of the covering layer can be further increased. As a result, a tube with higher durability can be provided.

Although each embodiment has been described, in the tube for an endoscope of the invention, the respective embodiments can be arbitrarily combined with each other in addition to independently Implementing each embodiment. For example, it is possible to provide a tube which has the covering layer formed of thermoplastic resin mixed with inorganic powder and silicone oil, the adhesive layer, and the surface-treated layer.

Further, in another aspect of the present invention, an endoscope equipped with the tube for an endoscope of the invention is provided.

In the endoscope of the invention, both ends of the tube for an endoscope are connected to a connector. FIG. 8A is a front view of an endoscope tip part 21. FIG. 8B is a cross-sectional view along arrow A-A in FIG. 8A. A forceps opening 22, an opening 23 for an imaging part, an opening 24 for an illumination part which are connected to the tube for an endoscope are provided at the center of the endoscope tip part 21.

FIG. 8C is a cross-sectional view for explaining connection between the tube 10 for an endoscope, connectors 9 aid 20, and the endoscope tip part 21. The end part of the tube 10 is connected to the connector 20 on the front end side. The connector 20 is connected to the forceps opening 22 of the endoscope tip part 21. The rear end part of the tube 10 is connected to the connector 9 on the rear end side.

In that case, in the tube 10 for an endoscope, hard wire rods forming the net layer 13 protrude from the cut surface at both ends of the tube 10. These protruding hard wire rods are preferably sealed with an adhesive agent 25, and more preferably fixed to the connectors 20 and 9. At this time, it is preferable that the adhesive agent 25 is applied so as to cover the protruding hard wire rods. According to this constitution, the tube and the connectors can be firmly connected to each other. As the adhesive agent 25 sealing the hard wire rods, an adhesive agent which is the same as or different from the adhesive agent forming the adhesive layer 16 may be used.

### EXAMPLES

### Example 1

### Investigation of Thickness and Durability of Covering Layer

The relation between the thickness of the covering layer and the durability thereof was examined. A tubular member (inner diameter: 2.6 mm) was covered by a net formed of a metal wire (outer diameter: 0.05 mm). The covering layer formed of nylon elastomer was formed on the net, and thus a tube with an outer diameter of 3.0 mm including the thickness of the covering layer was formed. In order to keep the outer diameter constant, when the covering layer had great thickness, the tubular member of small outer diameter was used, and when the covering layer had small thickness, the tubular member of great outer diameter was used.

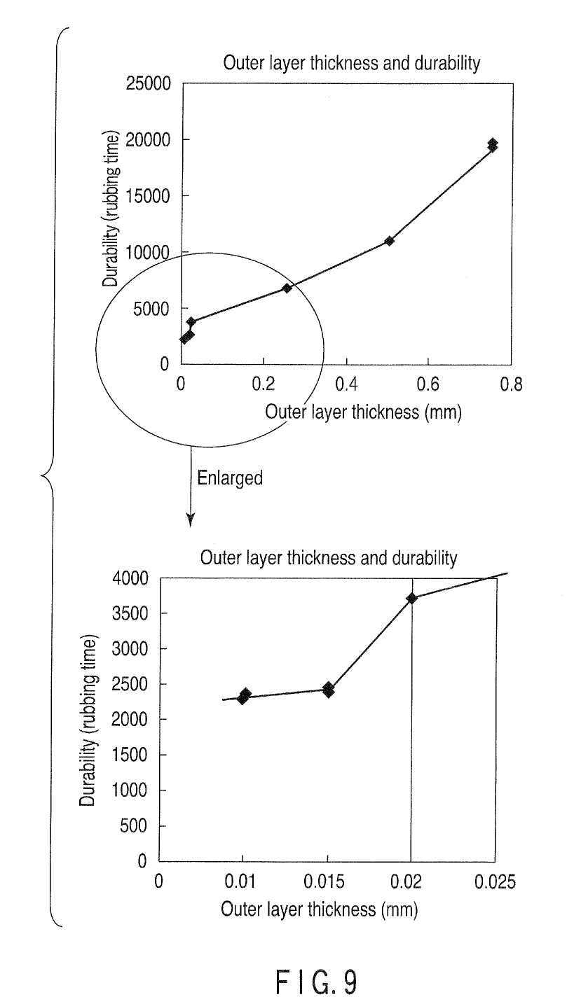

A metal pipe (outer diameter: 2.6 mm) was covered by the tube, and a wire provided with a weight of 500 g was suspended thereto. The wire had an outer diameter of 0.5 mm, and was a stranded wire formed of wires with an outer diameter of 0.25 mm. The wire was vertically moved in a width range of 10 mm, and rubbed against the covering layer until the surface of the metal wire was exposed. The frequency of rubbing was represented as the durability. The relation between the covering layer and the durability was shown in Table 1 and FIG. 9.

As shown in FIG. 9, it was shown that high durability was obtained when the thickness of the covering layer was not less than 0.02 mm.

TABLE 1

| Outer layer | Durability |
|---|---|
| thickness (mm) | (rubbing time) (times) |
| 0.01 | 2325 |
| 0.015 | 2417 |
| 0.02 | 3682 |
| 0.25 | 6784 |
| 0.5 | 11131 |
| 0.75 | 19562 |

### Example 2

### Investigation of Thickness of Covering Layer and Bending Force

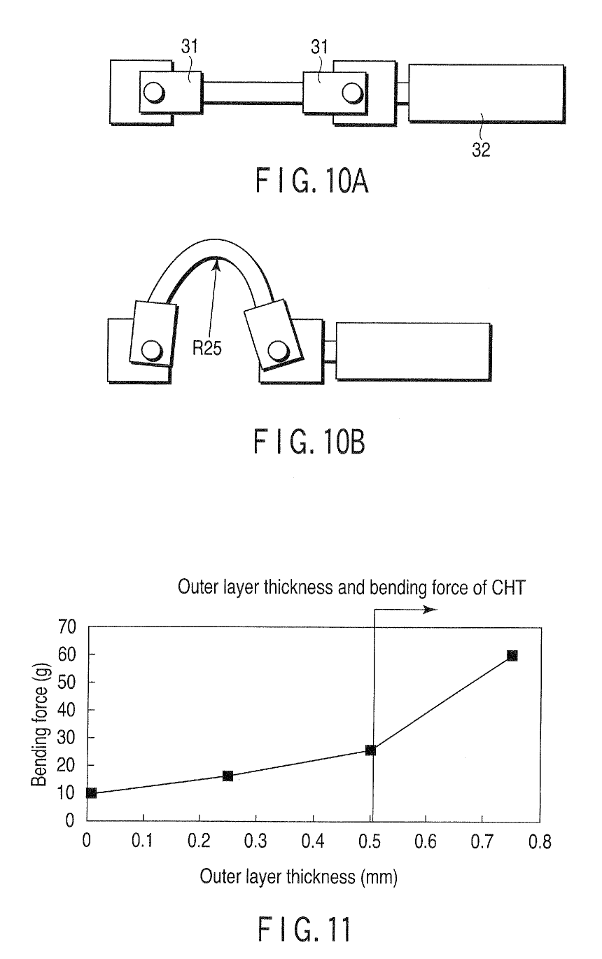

The relation between the thickness of the covering layer and the bending force was examined The same tube as in Example 1 was used. The thickness of the covering layer was changed from 0.01 to 0.75 mm. As shown in FIG. 10A, a tube was attached to an evaluation device. In FIG. 10A, reference number 31 indicates a member for attaching the tube and connected to a sensor 32 for measuring a reaction force of the curved tube. Next, as shown in FIG. 10B, the tube was bent so as to have a circular arc shape with a 25-mm radius (R25), and the bending force (g) at this time was measured.

The result is shown in Table 2 and FIG. 11. It was shown that when the thickness of the covering layer exceeded 0.5 mm, the bending force was rapidly increased. This shows that great force is required to curve the tube. Thus, this examination revealed that when the thickness of the covering layer exceeded 0.5 mm, a bending tube of an endoscope became less likely to bend, and thus the operability was reduced.

TABLE 2

| Outer layer | |
|---|---|
| thickness (mm) | Bending force (g) |
| 0.01 | 10.6 |
| 0.015 | 10.75 |
| 0.02 | 10.95 |
| 0.25 | 16.2 |
| 0.5 | 25.5 |
| 0.75 | 59.2 |

### Example 3

### Investigation of Thickness and Durability of Covering Layer Mixed with Inorganic Powder

As in the case of Example 1, the relation between the thickness of the covering layer and the durability thereof was examined. In this example, 30 parts by weight of barium sulfate with a particle diameter of 1 mm was mixed with 100 parts by weight of nylon elastomer in the formation of the covering layer. The result is shown in Table 3. As can be clearly seen in Table 3, it was shown that high durability can be obtained when the thickness of the covering layer was not less than 0.02 mm. Further, compared with Example 1, it was shown that higher durability could be obtained even when the covering layer had the same thickness as in Example 1.

TABLE 3

| Outer layer | Durability |
|---|---|
| thickness (mm) | (rubbing time) (times) |
| 0.01 | 2895 |
| 0.015 | 2958 |
| 0.02 | 4175 |
| 0.25 | 7735 |
| 0.5 | 11845 |
| 0.75 | 20089 |

### Example 4

### Investigation of Thickness and Durability of Covering Layer Mixed with Silicone Oil

As in the case of Example 1, the relation between the thickness of the covering layer and the durability thereof was examined. In this example, 10 parts by weight of silicone oil with a viscosity of 80000 CS was mixed with 100 parts by weight of nylon elastomer in the formation of the covering layer. The result is shown in Table 4. As can be clearly seen in Table 4, it was shown that high durability could be obtained when the thickness of the covering layer was not less than 0.02 mm. Further, compared with Example 1, it was found that higher durability could be obtained even when the covering layer had the same thickness as in Example 1.

TABLE 4

| Outer layer | Durability |
|---|---|
| thickness (mm) | (rubbing time) (times) |
| 0.01 | 3019 |
| 0.015 | 3103 |
| 0.02 | 4311 |
| 0.25 | 7880 |
| 0.5 | 12008 |
| 0.75 | 20221 |

### Example 5

### Investigation of Thickness and Durability of Covering Layer Formed on Adhesive Layer

As in the case of Example 1, the relation between the thickness of the covering layer and the durability thereof was examined. However, in this example, after an adhesive layer formed of epoxy-based adhesive was formed, the covering layer was formed. The result is shown in Table 5. As can be clearly seen in Table 5, it was shown that high durability could be obtained when the thickness of the covering layer was not less than 0.02 mm. Further, compared with Example 1, it was found that higher durability could be obtained even when the covering layer had the same thickness as in Example 1.

TABLE 5

| Outer layer | Durability |
|---|---|
| thickness (mm) | (rubbing time) (times) |
| 0.01 | 2427 |
| 0.015 | 2522 |
| 0.02 | 3805 |
| 0.25 | 6880 |
| 0.5 | 11234 |
| 0.75 | 19667 |

### Example 6

### Investigation of Thickness of Net Layer and Durability of Covering Layer

A tube was produced by using a metal wire (outer diameter: 0.05 mm) having a circular cross section and an elliptical metal wire (shorter outer diameter: 0.025 mm), respectively. The covering layer and the tubular member were formed of nylon elastomer. As in the above example, the covering layer was rubbed with a wire, and the relation between the thickness of the covering layer and the durability thereof was examined. The result is shown in Table 6.

TABLE 6

| | | | Wire of 0.05 mm | | Wire of 0.025 mm | |
|---|---|---|---|---|---|---|
| Outer diameter | Inner diameter | Inner layer | Outer layer | Durability | Outer layer | Durability |
| (mm) | (mm) | thickness (mm) | thickness (mm) | (rubbing time) | thickness (mm) | (rubbing time) |
| 3.0 | 2.6 | 0.09 | 0.01 | 2325 | 0.02 | 3694 |
| 3.0 | 2.6 | 0.085 | 0.015 | 2417 | 0.03 | 3882 |
| 3.0 | 2.6 | 0.08 | 0.02 | 3682 | 0.04 | 4018 |
| 3.0 | 2.6 | 0.05 | 0.05 | 3894 | 0.1 | 5024 |

The tubes used in the above examples have the same cuter and inner diameters. Thus, the smaller the thickness of the inner layer (tubular member), the greater the thickness of the outer layer (covering layer). Each inner layer thickness in the case of using the elliptical metal wire is greater than that in the case of using the circular metal wire. As a result, it was shown that the tube using the elliptical metal wire had greater durability than the tube using the circular metal wire.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A tube for an endoscope, comprising:
a tubular member forming a conduit line;
a net layer which covers an outside of the tubular member and is formed of a hard linear member; and
a covering layer which covers an outside of the net layer,
wherein the covering layer is formed of a material comprising thermoplastic resin and has a thickness ranging from 0.02 to 0.5 mm.

2. The tube for an endoscope according to claim 1, wherein the covering layer is formed of a material comprising thermoplastic resin and inorganic powder.

3. The tube for an endoscope according to claim 1, wherein the covering layer is formed of a material comprising thermoplastic resin and silicone oil.

4. The tube for an endoscope according to claim 1, wherein the covering layer is formed of a material comprising thermoplastic resin, inorganic powder, and silicone oil.

5. The tube for an endoscope according to claim 2, wherein the inorganic powder is selected from the group consisting of barium sulfate, carbon powder, boron nitride, molybdenum disulfide, silicon nitride, alumina, mica and mixtures thereof.

6. The tube for an endoscope according to claim 3, wherein the silicone oil has a viscosity ranging from 8500 to 1500000 CS.

7. The tube for an endoscope according to claim 1, further comprising an adhesive layer in the position between the net layer and the covering layer.

8. The tube for an endoscope according to claim 1, further comprising a surface-treated layer in the position between the net layer and the covering layer.

9. The tube for an endoscope according to claim 1, further comprising a surface-treated layer and an adhesive layer in the position between the net layer and the covering layer.

10. The tube for an endoscope according to claim 7, wherein the tubular member is formed of fluororesin or olefinic resin.

11. The tube for an endoscope according to claim 1, wherein the covering layer is formed of a material comprising thermoplastic resin and inorganic powder, and the tube further comprises at least ore layer which is provided between the net layer and the covering layer and selected from a surface-treated layer and an adhesive layer.

12. The tube for an endoscope according to claim 1, wherein the covering layer is formed of a material comprising thermoplastic resin and silicone oil, and the tube further comprises at least one layer selected from a surface-treated layer and an adhesive layer in the position between the net layer and the covering layer.

13. The tube for an endoscope according to claim 1, wherein the covering layer is formed of a material comprising thermoplastic resin, inorganic powder, and silicone oil, and the tube further comprises at least one layer selected from a surface-treated layer and an adhesive layer in the position between the net layer and the covering layer and.

14. The tube for an endoscope according to claim 1, wherein the net layer is formed of a hard linear member having an elliptical cross section, in which a diameter direction of the tubular member is a minor axis.

15. The tube for an endoscope according to claim 1, wherein the covering layer is formed of a material comprising thermoplastic resin and inorganic powder, and the net layer is formed of a hard linear member having an elliptical cross section, in which a diameter direction of the tubular member is a minor axis.

16. The tube for an endoscope according to claim 1, wherein the covering layer is formed of a material comprising thermoplastic resin and silicone oil, and the net layer is formed of a hard linear member having an elliptical cross section, in which a diameter direction of the tubular member is a minor axis.

17. The tube for an endoscope according to claim 1, wherein the covering layer is formed of a material comprising thermoplastic resin and inorganic powder, the tube further comprises at least one layer selected from a surface-treated layer and an adhesive layer in the position between the net layer and the covering layer, and the net layer is formed of a hard linear member having an elliptical cross section, in which a diameter direction of the tubular member is a minor axis.

18. The tube for an endoscope according to claim 1, wherein the covering layer is formed of a material comprising thermoplastic resin and silicone oil, the tube further comprises at least one layer selected from a surface-treated layer and an adhesive layer in the position between the net layer and the covering layer, and the net layer is formed of a hard linear member having an elliptical cross section, in which a diameter direction of the tubular member is a minor axis.

19. The tube for an endoscope according to claim 1, wherein the covering layer is formed of a material comprising thermoplastic resin, inorganic powder and silicone oil, the tube further comprises at least one layer selected from a surface-treated layer and an adhesive layer in the position between the net layer and the covering layer, and the net layer is formed of a hard linear member having an elliptical cross section, in which a diameter direction of the tubular member is a minor axis.

20. An endoscope comprising the tube for an endoscope according to claim 1.
